(19) United States
(12) Patent Application Publication
(10) Pub. No.: US 2007/0225276 A1
(43) Pub. Date: Sep. 27, 2007

(54) 7-Phenylsulfonyl-Tetrahydro-3-Benzazepine Dervatives as Antipsychotic Agents

(76) Inventors: Ian Thomson Forbes, Essex (GB); Gabriella Gentile, Verona (IT); Andrew Derrick Gribble, Essex (GB); Andrew P. Lightfoot, Essex (GB); Andrew H. Payne, Essex (GB); Alexander Baxter Smith, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/580,641

(22) PCT Filed: Nov. 25, 2004
(86) PCT No.: PCT/EP04/13418

§ 371 Date: Feb. 26, 2007

(30) Foreign Application Priority Data
Nov. 28, 2003 (GB) 0327741.5

(51) Int. Cl. A61K 31/55; C07D 223/16

(52) U.S. Cl. 514/217.01; 540/594

(57) ABSTRACT

The invention provides one or more chemical entities selected from a compound of formula (I): 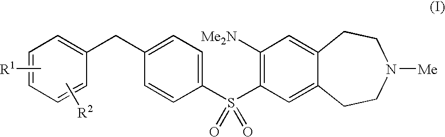 wherein
R¹ represents C₁₋₆alkyl, C₁₋₆alkoxy, trifluoromethyl, trifluoromethoxy, halo, cyano, 5-methyl-1,2,4-oxadiazol-3-yl or a group —SO₂X;
R² represents hydrogen, C₁₋₆alkyl, C₁₋₆alkoxy, halo or cyano;
X is C₁₋₆alkyl, —NR³R⁴ or morpholino;
R³ and R⁴ independently represent hydrogen or C₁₋₆alkyl; and a pharmaceutically acceptable salt and solvate thereof; with the proviso that the compound {8-[4-(4-fluoro-benzyl)-benzenesulfonyl]-3-methyl-2,3,4,5-tetrahydro-1H-3-benzazepin-7-yl}-dimethyl-amine is excluded. The compounds of formula (I) and pharmaceutically acceptable salts and solvates thereof are useful in therapy, in particular as antipsychotic agents.

This invention relates to novel compounds, pharmaceutical compositions containing them and their use in therapy, in particular as antipsychotic agents.

We have now found a novel group of phenylsulfonyl compounds which are useful particularly as antipsychotic agents.

The novel compounds of formula (I) have high affinities at desired receptors, exhibit good in vivo profiles and have good Drug Metabolism and Pharmacokinetics (DMPK) properties.

In a first aspect of the invention, there is provided one or more chemical entities selected from a compound of formula (I): 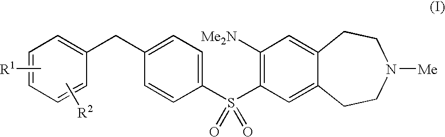
wherein
R¹ represents C₁₋₆alkyl, C₁₋₆alkoxy, trifluoromethyl, trifluoromethoxy, halo, cyano, 5-methyl-1,2,4-oxadiazol-3-yl or a group —SO₂X;
R² represents hydrogen, C₁₋₆alkyl, C₁₋₆alkoxy, halo or cyano;
X represents C₁₋₆alkyl, —NR³R⁴ or morpholino;
R³ and R⁴ independently represent hydrogen or C₁₋₆alkyl;
and a pharmaceutically acceptable salt and solvate thereof;
with the proviso that the compound {8-[4-(4-fluoro-benzyl)-benzenesulfonyl]-3-methyl-2,3,4,5-tetrahydro-1H-3-benzazepin-7-yl}-dimethyl-amine is excluded.

As used herein, the term “alkyl” refers to straight or branched hydrocarbon chains containing the specified number of carbon atoms. For example, C₁₋₆alkyl means a straight or branched alkyl containing at least 1, and at most 6, carbon atoms. Examples of “alkyl” as used herein include, but are not limited to, methyl, ethyl, n-propyl, n-butyl, n-pentyl, n-hexyl, isobutyl, isopropyl, t-butyl and 1,1-dimethylpropyl.

As used herein, the term “alkoxy” refers to a straight or branched alkoxy group containing the specified number of carbon atoms. For example, C₁₋₆alkoxy means a straight or branched alkoxy group containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy or hexyloxy.

As used herein, the term “halo” refers to fluoro, chloro, bromo and iodo.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include water, methanol, ethanol and acetic acid. Most preferably the solvent used is water and the solvate may also be referred to as a hydrate.

It will be appreciated that for use in medicine the salts of formula (I) should be pharmaceutically acceptable. Suitable pharmaceutically acceptable salts will be apparent to those skilled in the art and include for example acid addition salts formed with inorganic acids e.g. hydrochloric, hydrobromic, sulfuric, nitric or phosphoric acid; and organic acids e.g. succinic, maleic, malic, mandelic, acetic, fumaric, glutamic, lactic, citric, tartaric, benzoic, benzenesulfonic, p-toluenesulfonic, methanesulfonic or naphthalenesulfonic acid. Examples of salts include the hydrochloride, maleate, tosylate or mesylate salts or pharmaceutically acceptable derivatives thereof. Other non-physiologically acceptable salts e.g. oxalates, may be used, for example in the isolation of compounds of formula (I) and are included within the scope of this invention. Also included within the scope of the invention are solvates and hydrates of the compounds of formula (I).

Certain of the compounds of formula (I) may form acid addition salts with one or more equivalents of the acid. The present invention includes within its scope all possible stoichiometric and non-stoichiometric forms thereof.

Certain salts of compounds of formula (I) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the salts of compounds represented by formula (I) as mixtures with isomers thereof in which one or more chiral centres are inverted. Likewise, it is understood that salts of compounds of formula (I) may exist in tautomeric forms other than that shown in the formula and these are also included within the scope of the present invention.

The term “pharmaceutically acceptable derivative” as used herein refers to any pharmaceutically acceptable derivative of a compound of the present invention, for example, an ester, which upon administration to a mammal, such as a human, is capable of providing (directly or indirectly) such a compound or an active metabolite thereof. Such derivatives are clear to those skilled in the art, without undue experimentation, and with reference to the teaching of Burger's Medicinal Chemistry And Drug Discovery, 5th Edition, Vol 1: Principles And Practice, which is incorporated herein by reference.

In a further aspect of the invention, R¹ represents C₁₋₆alkyl for example methyl (such as 2-methyl, 3-methyl, or 4-methyl); C₁₋₆alkoxy for example methoxy (such as 2-methoxy, 3-methoxy or 4-methoxy) or ethoxy (such as 4-ethoxy); trifluoromethoxy (such as 3-trifluoromethoxy or 4-trifluoromethoxy); halo for example fluoro (such as 2-fluoro, 3-fluoro, 4-fluoro or 5-fluoro) or chloro (such as 2-chloro, 3-chloro or 4-chloro); trifluoromethyl (such as 2-trifluoromethyl, 3-trifluoromethyl, 4-trifluoromethyl or 5-trifluoromethyl); cyano (such as 2-cyano, 3-cyano or 4-cyano); methanesulfonyl (such as 4-methanesulfonyl); N,N-dimethylaminosulfonyl [such as 4-(N,N-dimethylaminosulfonyl)]; morpholinosulfonyl (such as 4-morpholinosulfonyl) or 5-methyl-1,2,4-oxadiazol-3-yl [such as 4-(5-methyl-1,2,4-oxadiazol-3-yl)].

In another aspect of the invention, R² represents hydrogen; halo for example fluoro (such as 2-fluoro, 3-fluoro, 4-fluoro or 5-fluoro) or chloro (such as 2-chloro or 4-chloro); cyano for example 2-cyano, 3-cyano or 4-cyano; C₁₋₆alkyl for example methyl (such as 2-methyl or 4-methyl); or C₁₋₆alkoxy for example methoxy (such as 3-methoxy or 4-methoxy).

When R² is hydrogen, R¹ may be C₁₋₆alkyl for example methyl (such as 2-methyl, 3-methyl or 4-methyl); C₁₋₆alkoxy for example methoxy (such as 2-methoxy, 3-methoxy or 4-methoxy) or ethoxy (such as 4-ethoxy); trifluoromethoxy such as 3-trifluoromethoxy or 4-trifluoromethoxy; halo for example fluoro (such as 2-fluoro or 3-fluoro) or chloro (such as 2-chloro, 3-chloro or 4-chloro); trifluoromethyl for example 3-trifluoromethyl; cyano such as 2-cyano, 3-cyano or 4-cyano; methanesulfonyl such as 4-methanesulfonyl; N,N-dimethylaminosulfonyl such as 4-(N,N-dimethylaminosulfonyl); morpholinosulfonyl such as 4-morpholinosulfonyl or 5-methyl-1,2,4-oxadiazol-3-yl such as 4-(5-methyl-1,2,4-oxadiazol-3-yl).

When R² is other than hydrogen as described hereinbefore, the combination of R¹ and R² substituents may be dihalo for example dichloro (such as 2,4-dichloro or 3,4-dichloro), difluoro (such as 2,4-difluoro, 2,5-difluoro, 3,4-difluoro or 3,5-difluoro), and chloro and fluoro (such as 2-chloro-4-fluoro, 3-chloro-4-fluoro or 3-chloro-5-fluoro); trifluoromethyl and halo, for example trifluoromethyl and fluoro (such as 3-trifluoromethyl-4-fluoro, 3-trifluoromethyl-5-fluoro, 3-fluoro-5-trifluoromethyl, 2-fluoro-5-trifluoromethyl, 2-trifluoromethyl-4-fluoro, 2-fluoro-4-trifluoromethyl or 2-trifluoromethyl-5-fluoro), or trifluoromethyl and chloro (such as 3-trifluoromethyl-4-chloro, 2-chloro-3-trifluoromethyl or 2-chloro-5-trifluoromethyl); trifluoromethyl and cyano (such as 2-trifluoromethyl-4-cyano); halo and C₁₋₆alkyl for example fluoro and methyl (such as 2-fluoro-3-methyl, 3-fluoro-4-methyl, 2-methyl-5-fluoro or 3-methyl-4-fluoro); cyano and halo, for example cyano and fluoro (such as 2-fluoro-4-cyano, 5-fluoro-3-cyano, 4-fluoro-2-cyano, 4-fluoro-3-cyano or 3-fluoro-4-cyano), or cyano and chloro (such as 2-chloro-4-cyano); trifluoromethyl and C₁₋₆alkoxy, for example trifluoromethyl and methoxy (such as 3-methoxy-4-trifluoromethyl); cyano and C₁₋₆alkoxy, for example cyano and methoxy (such as 3-cyano-4-methoxy); or trifluoromethoxy and halo for example trifluoromethoxy and fluoro (such as 3-fluoro-4-trifluoromethyl).

In a yet further aspect of the invention, R¹ represents 2-methyl, 3-methyl, 4-methyl, 2-methoxy, 3-methoxy, 4-methoxy 4-ethoxy, 3-trifluoromethoxy, 4-trifluoromethoxy, 2-fluoro, 3-fluoro, 4-fluoro, 5-fluoro, 2-chloro, 3-chloro, 4-chloro, 2-trifluoromethyl, 3-trifluoromethyl, 4-trifluoromethyl, 5-trifluoromethyl, 2-cyano, 3-cyano, 4-cyano, 4-methanesulfonyl, 4-(N,N-dimethylaminosulfonyl), 4-morpholinosulfonyl or 4-(5-methyl-1,2,4-oxadiazol-3-yl).

In another aspect of the invention, R² represents hydrogen, 2-fluoro, 3-fluoro, 4-fluoro, 5-fluoro, 2-chloro, 4-chloro, 2-cyano, 3-cyano, 4-cyano, 2-methyl, 4-methyl, 3-methoxy or 4-methoxy.

When R² is hydrogen, R¹ may be 2-methyl, 3-methyl, 4-methyl, 2-methoxy, 3-methoxy, 4-methoxy, 4-ethoxy, 3-trifluoromethoxy, 4-trifluoromethoxy, 2-fluoro, 3-fluoro, 2-chloro, 3-chloro, 4-chloro, 3-trifluoromethyl, 2-cyano, 3-cyano, 4-cyano, 4-methanesulfonyl, 4-(N,N-dimethylaminosulfonyl), 4-morpholinosulfonyl or 4-(5-methyl-1,2,4-oxadiazol-3-yl).

When R² is other than hydrogen as described hereinbefore, the combination of R¹ and R² substituents may be 2,4-dichloro, 3,4-dichloro, 2,4-difluoro, 2,5-difluoro, 3,4-difluoro, 3,5-difluoro, 3-trifluoromethyl-4-fluoro, 3-trifluoromethyl-5-fluoro, 3-fluoro-5-trifluoromethyl, 3-trifluoromethyl-4-chloro, 2-fluoro-5-trifluoromethyl, 2-trifluoromethyl-4-fluoro, 2-trifluoromethyl-4-cyano, 2-fluoro-4-trifluoromethyl, 2-chloro-3-trifluoromethyl, 2-trifluoromethyl-5-fluoro, 2-chloro-5-trifluoromethyl, 2-chloro-4-fluoro, 3-chloro-4-fluoro, 2-fluoro-3-methyl, 3-fluoro-4-methyl, 2-methyl-5-fluoro, 3-methyl-4-fluoro, 3-chloro-5-fluoro, 2-chloro-4-cyano, 2-fluoro-4-cyano, 3-methoxy-4-trifluoromethyl, 5-fluoro-3-cyano, 4-fluoro-2-cyano, 4-fluoro-3-cyano, 3-fluoro-4-cyano, 3-cyano-4-methoxy or 3-fluoro-4-trifluoromethyl.

In a further aspect of the invention, there is provided one or more chemical entities selected from a compound of formula (I) as described hereinbefore wherein

R¹ represents methyl, trifluoromethyl, methoxy, ethoxy, trifluoromethoxy, fluoro, chloro, cyano, methanesulfonyl or 5-methyl-1,2,4-oxadiazol-3-yl;

R² represents hydrogen, methyl, fluoro, chloro, cyano or methoxy;

and a pharmaceutically acceptable salt and solvate thereof;

with the proviso that the compound {8-[4-(4-fluoro-benzyl)-benzenesulfonyl]-3-methyl-2,3,4,5-tetrahydro-1H-3-benzazepin-7-yl}-dimethyl-amine is excluded.

It is to be understood that the present invention covers all combinations of particular and preferred groups described herein above.

In a further aspect of the invention, there are provided one or more chemical entities selected from compounds including those incorporated in Table 1 and those specifically exemplified and named hereinafter including, without limitation:—
{8-[4-(3-Fluoro-5-trifluoromethyl-benzyl)-benzenesulfonyl]-3-methyl-2,3,4,5-tetrahydro-1H-3-benzazepin-7-yl}-dimethyl-amine;
{8-[4-(4-Cyanobenzyl)benzenesulfonyl]-3-methyl-2,3,4,5-tetrahydro-1H-3-benzazepin-7-yl}-dimethylamine;
{8-[4-(2,4-Dichlorobenzyl)benzenesulfonyl]-3-methyl-2,3,4,5-tetrahydro-1H-3-benzazepin-7-yl}-dimethylamine;
{8-[4-(3-Trifluoromethylbenzyl)benzenesulfonyl]-3-methyl-2,3,4,5-tetrahydro-1H-3-benzazepin-7-yl}-dimethylamine;
{8-[4-(4-Ethoxybenzyl)benzenesulfonyl]-3-methyl-2,3,4,5-tetrahydro-1H-3-benzazepin-7-yl}-dimethylamine;
{8-[4-(4-Fluoro-3-trifluoromethylbenzyl)benzenesulfonyl]-3-methyl-2,3,4,5-tetrahydro-1H-3-benzazepin-7-yl}-dimethylamine;
{8-[4-(2-Methoxybenzyl)benzenesulfonyl]-3-methyl-2,3,4,5-tetrahydro-1H-3-benzazepin-7-yl}-dimethylamine;
{8-[4-(3-Fluorobenzyl)benzenesulfonyl]-3-methyl-2,3,4,5-tetrahydro-1H-3-benzazepin-7-yl}-dimethylamine;
{8-[4-(4-Methanesulfonylbenzyl)benzenesulfonyl]-3-methyl-2,3,4,5-tetrahydro-1H-3-benzazepin-7-yl}-dimethylamine;
{8-[4-(3-Methoxy-4-trifluoromethylbenzyl)benzenesulfonyl]-3-methyl-2,3,4,5-tetrahydro-1H-3-benzazepin-7-yl}-dimethylamine;
{8-[4-(3,5-Difluorobenzyl)benzenesulfonyl]-3-methyl-2,3,4,5-tetrahydro-1H-3-benzazepin-7-yl}-dimethylamine;
{8-[4-(2-Fluoro-5-trifluoromethylbenzyl)benzenesulfonyl]-3-methyl-2,3,4,5-tetrahydro-1H-3-benzazepin-7-yl}-dimethylamine;
{8-[4-(2-Trifluoromethyl-4-fluorobenzyl)benzenesulfonyl]-3-methyl-2,3,4,5-tetrahydro-1H-3-benzazepin-7-yl}-dimethylamine;
{8-[4-(2-Chloro-3-trifluoromethylbenzyl)benzenesulfonyl]-3-methyl-2,3,4,5-tetrahydro-1H-3-benzazepin-7-yl}-dimethylamine;
{8-[4-(2-Trifluoromethyl-5-fluorobenzyl)benzenesulfonyl]-3-methyl-2,3,4,5-tetrahydro-1H-3-benzazepin-7-yl}-dimethylamine;
{8-[4-(2-Chloro-5-trifluoromethylbenzyl)benzenesulfonyl]-3-methyl-2,3,4,5-tetrahydro-1H-3-benzazepin-7-yl}-dimethylamine;
{8-[4-(2,4-Difluorobenzyl)benzenesulfonyl]-3-methyl-2,3,4,5-tetrahydro-1H-3-benzazepin-7-yl}-dimethylamine;
{8-[4-(2-Chloro-4-fluorobenzyl)benzenesulfonyl]-3-methyl-2,3,4,5-tetrahydro-1H-3-benzazepin-7-yl}-dimethylamine;
{8-[4-(3-Chloro-4-fluorobenzyl)benzenesulfonyl]-3-methyl-2,3,4,5-tetrahydro-1H-3-benzazepin-7-yl}-dimethylamine;
{8-[4-(3-Trifluoromethoxybenzyl)benzenesulfonyl]-3-methyl-2,3,4,5-tetrahydro-1H-3-benzazepin-7-yl}-dimethylamine;
{8-[4-(2,5-Difluorobenzyl)benzenesulfonyl]-3-methyl-2,3,4,5-tetrahydro-1H-3-benzazepin-7-yl}-dimethylamine;
{8-[4-(2-Fluoro-3-methylbenzyl)benzenesulfonyl]-3-methyl-2,3,4,5-tetrahydro-1H-3-benzazepin-7-yl}-dimethylamine;
{8-[4-(2-Methyl-5-fluorobenzyl)benzenesulfonyl]-3-methyl-2,3,4,5-tetrahydro-1H-3-benzazepin-7-yl}-dimethylamine;
{8-[4-(3-Methyl-4-fluorobenzyl)benzenesulfonyl]-3-methyl-2,3,4,5-tetrahydro-1H-3-benzazepin-7-yl}-dimethylamine;
{8-[4-(3-Chloro-5-fluorobenzyl)benzenesulfonyl]-3-methyl-2,3,4,5-tetrahydro-1H-3-benzazepin-7-yl}-dimethylamine;
{8-[4-(3-Fluoro-4-trifluoromethylbenzyl)benzenesulfonyl]-3-methyl-2,3,4,5-tetrahydro-1H-3-benzazepin-7-yl}-dimethylamine;
{8-[4-(3,4-Difluorobenzyl)benzenesulfonyl]-3-methyl-2,3,4,5-tetrahydro-1H-3-benzazepin-7-yl}-dimethylamine;
{8-[4-(3-Methoxybenzyl)benzenesulfonyl]-3-methyl-2,3,4,5-tetrahydro-1H-3-benzazepin-7-yl}-dimethylamine;
{8-[4-(4-Methoxybenzyl)benzenesulfonyl]-3-methyl-2,3,4,5-tetrahydro-1H-3-benzazepin-7-yl}-dimethylamine;
{8-[4-(2-Methylbenzyl)benzenesulfonyl]-3-methyl-2,3,4,5-tetrahydro-1H-3-benzazepin-7-yl}-dimethylamine;
{8-[4-(3-Methylbenzyl)benzenesulfonyl]-3-methyl-2,3,4,5-tetrahydro-1H-3-benzazepin-7-yl}-dimethylamine;
{8-[4-(4-Methylbenzyl)benzenesulfonyl]-3-methyl-2,3,4,5-tetrahydro-1H-3-benzazepin-7-yl}-dimethylamine;
{8-[4-(2-Chlorobenzyl)benzenesulfonyl]-3-methyl-2,3,4,5-tetrahydro-1H-3-benzazepin-7-yl}-dimethylamine;
{8-[4-(3-Chlorobenzyl)benzenesulfonyl]-3-methyl-2,3,4,5-tetrahydro-1H-3-benzazepin-7-yl}-dimethylamine;
{8-[4-(4-Chlorobenzyl)benzenesulfonyl]-3-methyl-2,3,4,5-tetrahydro-1H-3-benzazepin-7-yl}-dimethylamine;
{8-[4-(2-Fluorobenzyl)benzenesulfonyl]-3-methyl-2,3,4,5-tetrahydro-1H-3-benzazepin-7-yl}-dimethylamine;
{8-[4-(3-Cyanobenzyl)benzenesulfonyl]-3-methyl-2,3,4,5-tetrahydro-1H-3-benzazepin-7-yl}-dimethylamine;
{8-[4-(3,4-Dichlorobenzyl)benzenesulfonyl]-3-methyl-2,3,4,5-tetrahydro-1H-3-benzazepin-7-yl}-dimethylamine;
{8-[4-(3-Fluoro-4-methylbenzyl)benzenesulfonyl]-3-methyl-2,3,4,5-tetrahydro-1H-3-benzazepin-7-yl}-dimethylamine;
{8-[4-(2-Cyanobenzyl)benzenesulfonyl]-3-methyl-2,3,4,5-tetrahydro-1H-3-benzazepin-7-yl}-dimethylamine;
{8-[4-(4-Trifluoromethoxybenzyl)benzenesulfonyl]-3-methyl-2,3,4,5-tetrahydro-1H-3-benzazepin-7-yl}-dimethylamine;
{8-[4-(3-Trifluoromethyl-5-fluorobenzyl)benzenesulfonyl]-3-methyl-2,3,4,5-tetrahydro-1H-3-benzazepin-7-yl}-dimethylamine;
{8-[4-(2-Chloro-4-cyanobenzyl)benzenesulfonyl]-3-methyl-2,3,4,5-tetrahydro-1H-3-benzazepin-7-yl}-dimethylamine;
{8-[4-(2-Fluoro-4-cyanobenzyl)benzenesulfonyl]-3-methyl-2,3,4,5-tetrahydro-1H-3-benzazepin-7-yl}-dimethylamine;
{8-[4-(4-Trifluoromethyl-2-fluorobenzyl)benzenesulfonyl]-3-methyl-2,3,4,5-tetrahydro-1H-3-benzazepin-7-yl}-dimethylamine;
{8-[4-(3-Trifluoromethyl-4-chlorobenzyl)benzenesulfonyl]-3-methyl-2,3,4,5-tetrahydro-1H-3-benzazepin-7-yl}-dimethylamine;
{8-[4-(2-Trifluoromethyl-4-cyanobenzyl)benzenesulfonyl]-3-methyl-2,3,4,5-tetrahydro-1H-3-benzazepin-7-yl}-dimethylamine;
{8-[4-(5-Methyl-1,2,4-oxadiazol-3-ylbenzyl)benzenesulfonyl]-3-methyl-2,3,4,5-tetrahydro-1H-3-benzazepin-7-yl}-dimethylamine;
3-[(4-{[8-(Dimethylamino)-3-methyl-2,3,4,5-tetrahydro-1H-3-benzazepin-7-yl]sulfonyl}phenyl)methyl]-5-fluorobenzonitrile;
2-[(4-{[8-(Dimethylamino)-3-methyl-2,3,4,5-tetrahydro-1H-3-benzazepin-7-yl]sulfonyl}phenyl)methyl]-5-fluorobenzonitrile;
5-[(4-{[8-(Dimethylamino)-3-methyl-2,3,4,5-tetrahydro-1H-3-benzazepin-7-yl]sulfonyl}phenyl)methyl]-2-fluorobenzonitrile;
4-[(4-{[8-(Dimethylamino)-3-methyl-2,3,4,5-tetrahydro-1H-3-benzazepin-7-yl]sulfonyl}phenyl)methyl]-2-fluorobenzonitrile;
4-[(4-{[8-(Dimethylamino)-3-methyl-2,3,4,5-tetrahydro-1H-3-benzazepin-7-yl]sulfonyl}phenyl)methyl]-N,N-dimethylbenzenesulfonamide;
5-[(4-{[8-(Dimethylamino)-3-methyl-2,3,4,5-tetrahydro-1H-3-benzazepin-7-yl]sulfonyl}phenyl)methyl]-2-(methyloxy)benzonitrile or
N,N,3-Trimethyl-8-[(4-{[4-(4-morpholinylsulfonyl)phenyl]methyl}phenyl)sulfonyl]-2,3,4,5-tetrahydro-1H-3-benzazepin-7-amine,
   and pharmaceutically acceptable salts and solvates thereof.

The present invention also provides a general process for preparing a compound of formula (I) which process comprises:
coupling a compound of formula (II) 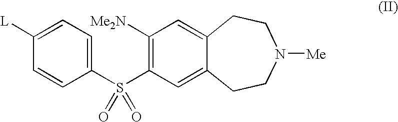
with a compound of formula (III) 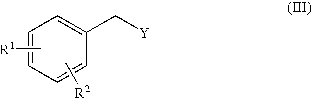
wherein L is a leaving group, such as bromo, and Y is an organozinc reagent such as ZnBr or ZnCl, and R¹ and R² are as hereinbefore described, in the presence of a suitable coupling reagent, such as palladium tetrakis(triphenylphosphine); and thereafter optionally for the above process:
converting a compound of formula (I) into another compound of formula (I); or
forming a pharmaceutically acceptable salt or solvate.

An example of the above general process is:

L is Br and Y is ZnBr and the reaction is conveniently carried out in an inert solvent, for example tetrahydrofuran, in the presence of palladium tetrakis(triphenylphosphine), optionally at elevated temperature, for example, 70° C.

Compounds of formula (II) may be prepared by deprotecting a compound of formula (IV) 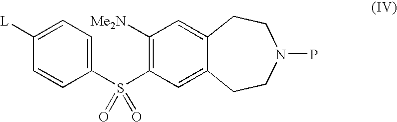
wherein L is a leaving group, such as bromo and P is a suitable protecting group, such as t-butoxycarbonyl (BOC), by using suitable deprotecting reagents, for example hydrochloric acid in dioxan, followed by reductive methylation using, for example, formaldehyde and a suitable reducing agent such as sodium triacetoxyborohydride. The reaction can be carried out in a suitable solvent, for example, 1,2-dichloroethane.

Compounds of formula (III) are commercially available, for example, 4-cyanobenzylzinc bromide (Rieke), or can be prepared using standard methods, for example using the method described by Knochel et al., J. Org. Chem. 1998, Vol. 53, p 5789-5791. The benzyl bromide precursor, if not commercially available, can be prepared by bromination of the corresponding toluene using standard methods, for example using the method described by Gilbert et al., J. Med. Chem. 2000, Vol. 43, p 1203-1214.

Compounds of formula (IV) may be prepared by oxidising a compound of formula (V) 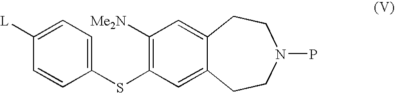
wherein L is a leaving group, such as bromo and P is a suitable protecting group, such as t-butoxycarbonyl (BOC), with an S-oxidising agent, such as magnesium monoperoxyphthalate hexahydrate (Aldrich). The reaction can be carried out in a suitable solvent, such as a mixture of dichloromethane and methanol.

Compounds of formula (V) may be prepared by reacting a compound of formula (VI) 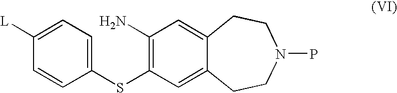
wherein L is a leaving group, such as bromo and P is a suitable protecting group, such as t-butoxycarbonyl (BOC), under reductive methylation conditions with aqueous formaldehyde and a suitable reducing agent such as sodium triacetoxyborohydride. The reaction can be carried out in a suitable solvent, such as dichloromethane, at room temperature.

Compounds of formula (VI) may be prepared by reducing a compound of formula (VII) 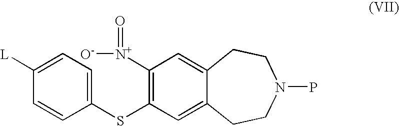
wherein L is a leaving group, such as bromo and P is a suitable protecting group, such as t-butoxycarbonyl (BOC), using a suitable reducing agent, such as iron, in the presence of an acid, for example acetic acid. The reaction can be carried out in a suitable solvent, such as ethanol, at an elevated temperature, for example 110° C.

Compounds of formula (VII) may be prepared by reacting a compound of formula (VIII) 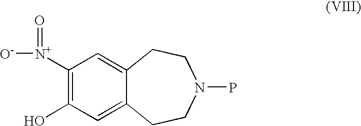
with a compound of formula (IX) 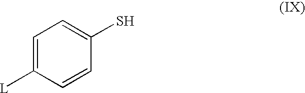
wherein L is a leaving group, such as bromo and P is a suitable protecting group, such as t-butoxycarbonyl (BOC). The OH function may be converted to a leaving group, for example a trifluoromethanesulfonate group. Conversion of the OH function to trifluoromethanesulfonate may be carried out by treatment with a trifluoromethanesulfonylating agent such as trifluoromethanesulfonyl chloride in the presence of a base, for example ethyl-diisopropylamine. The reaction of compounds of structure (VIII) with compounds of structure (IX) can be carried out in the presence of a suitable base, for example ethyl-diisopropylamine, in a suitable solvent, such as acetonitrile, at room temperature.

Compounds of formula (VIII) may be prepared by nitrating a compound of formula (X) 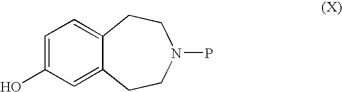
wherein P is a suitable protecting group, such as t-butoxycarbonyl (BOC), with a suitable nitrating agent, for example nitric acid. The reaction can be carried out in a suitable solvent, such as dichloromethane, at 0° C.

Compounds of formula (IX) are commercially available, for example 4-bromobenzenethiol (Aldrich) or may be prepared using literature methods.

Compounds of formula (X) may be prepared by using literature methods, for example that described by Hadley et al., Bioorg. Med. Chem. Lett.; 2000, 10, p 2553-2556 and previously described in patent EP285287.

As used herein, the term “suitable protecting group” refers to groups that are described in many standard texts on organic chemistry, for example in ‘Protective Organic Synthesis’, P. W. Green, Wiley 1981. Examples of suitable protecting groups include, but are not limited to, t-butoxycarbonyl (BOC).

In a further aspect of the invention, there is provided a process to prepare a compound of formula (I) as hereinbefore defined by coupling a compound of formula (II) 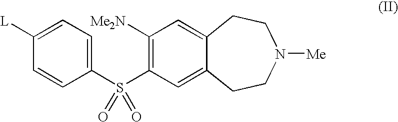
with a compound of formula (III) 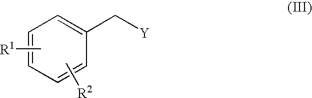
wherein L is a leaving group, Y is an organozinc reagent and R¹ and R² are as hereinbefore described, in the presence of a suitable coupling reagent; and thereafter optionally for the above process:
converting a compound of formula (I) into another compound of formula (I); or
forming a pharmaceutically acceptable salt or solvate.

Certain compounds of formula (I) and their pharmaceutically acceptable salts and solvates thereof have been found to exhibit affinity for dopamine receptors, in particular the D₃ and D₂ receptors, and are useful in the treatment of disease states which require modulation of such receptors, such as psychotic conditions. Many of the compounds of formula (I) have also been found to have greater affinity for dopamine D₃ than for D₂ receptors. The therapeutic effect of currently available antipsychotic agents (neuroleptics) is generally believed to be exerted via blockade of D₂ receptors; however this mechanism is also thought to be responsible for undesirable eps associated with many neuroleptic agents. Without wishing to be bound by theory, it has been suggested that blockade of the dopamine D₃ receptor may give rise to beneficial antipsychotic activity without significant extrapyramidal side effects (eps) (see for example Sokoloff et al., Nature, 1990; 347: 146-151; and Schwartz et al., Clinical Neuropharmacology, Vol 16, No. 4, 295-314, 1993).

Compounds of formula (I) and their pharmaceutically acceptable salts and solvates thereof also have antagonist affinity for the serotonin 5-HT_{2C}, 5-HT_{2A} and 5-HT₆ receptors. These properties may give rise to antipsychotic activity (e.g. improved effects on cognitive dysfunction), activity with reduced extrapyramidal side effects (eps), and/or anxiolytic/antidepressant activity. These could include, but are not limited to, attenuation of cognitive symptoms via 5-HT₆ receptor blockade (see Reavill, C. and Rogers, D.C., 2001, Investigational Drugs 2, 104-109), and reduced anxiety (see for example Kennett et al., Neuropharmacology 1997 April-May; 36 (4-5): 609-20), protection against eps (Reavill et al., Brit. J. Pharmacol., 1999; 126: 572-574) and antidepressant activity (Bristow et al., Neuropharmacology 39:2000; 1222-1236) via 5-HT_{2C} receptor blockade.

Compounds of formula (I) and their pharmaceutically acceptable salts and solvates thereof may also exhibit affinity for other receptors not mentioned above, resulting in beneficial antipyschotic activity.

The compounds of formula (I) and their pharmaceutically acceptable salts and solvates thereof are of use as antipsychotic agents for example in the treatment of schizophrenia, schizo-affective disorders, schizophreniform diseases, psychotic depression, mania, acute mania, paranoid and delusional disorders. Furthermore, they may have utility as adjunct therapy in Parkinsons Disease, particularly with compounds such as L-DOPA and possibly dopaminergic agonists, to reduce the side effects experienced with these treatments on long term use (e.g. see Schwartz et al., Brain Res. Reviews, 1998, 26, 236-242). From the localisation of D₃ receptors, it could also be envisaged that the compounds could also have utility for the treatment of substance abuse where it has been suggested that D₃ receptors are involved (e.g. see Levant, 1997, Pharmacol. Rev., 49, 231-252). Examples of such substance abuse agents include cocaine, ethanol, nicotine, benzodiazepines, alcohol, caffeine, phencyclidine and phencyclidine-like compounds, opiates such as cannabis, heroin, morphine, sedative ipnotic, amphetamine or amphetamine-related drugs such as dextroamphetamine, methylamphetamine or a combination thereof. Other conditions which may be treated by the compounds include dyskinetic disorders such as Parkinson's disease, neuroleptic-induced parkinsonism and tardive dyskinesias; depression (which term includes bipolar depression, unipolar depression, single or recurrent major depressive episodes with or without psychotic features, catatonic features, melancholic features, atypical features or postpartum onset, seasonal affective disorder and dysthymia, depressive disorders resulting from a general medical condition including, but not limited to, myocardial infarction, diabetes, miscarriage or abortion); anxiety disorders (which includes generalised anxiety and social anxiety disorder); agitation; tension; social or emotional withdrawal in psychotic patients; cognitive impairment including memory disorders such as Alzheimer's disease; psychotic states associated with neurodegenerative disorders, e.g. Alzheimer's disease; eating disorders (including anorexia nervosa and bulimia nervosa); obesity; sexual dysfunction; sleep disorders (including disturbances of circadian rhythm, dyssomnia, insomnia, sleep apnea and narcolepsy); emesis; movement disorders; obsessive-compulsive disorders; amnesia; aggression; autism; vertigo; dementia; circadian rhythm disorders; convulsions; epilepsy; and gastric motility disorders e.g. IBS.

A compound of formula (I) as hereinbefore described or a pharmaceutically acceptable salt and solvate thereof may be of use in the treatment of psychotic disorders.

In a further aspect therefore, the invention provides one or more chemical entities selected from a compound of formula (I) and a pharmaceutically acceptable salt and solvate thereof for use in therapy.

In another aspect, the invention provides one or more chemical entities selected from a compound of formula (I) and a pharmaceutically acceptable salt and solvate thereof for use in the treatment of a condition which requires modulation of a dopamine receptor.

In a further aspect, the invention provides one or more chemical entities selected from a compound of formula (I) and a pharmaceutically acceptable salt and solvate thereof for use in the treatment of psychotic disorders.

In another aspect, the invention provides the use of one or more chemical entities selected from a compound of formula (I) and a pharmaceutically acceptable salt and solvate thereof in the manufacture of a medicament for the treatment of a condition which requires modulation of a dopamine receptor.

In a further aspect, the invention provides the use of one or more chemical entities selected from a compound of formula (I) and a pharmaceutically acceptable salt and solvate thereof in the manufacture of a medicament for the treatment of psychotic disorders.

In another aspect, the invention provides a method of treating a condition which requires modulation of a dopamine receptor, which comprises administering to a mammal in need thereof an effective amount of one or more chemical entities selected from a compound of formula (I) and a pharmaceutically acceptable salt and solvate thereof.

In a further aspect, the invention provides a method of treating psychotic disorders which comprises administering to a mammal in need thereof an effective amount of one or more chemical entities selected from a compound of formula (I) and a pharmaceutically acceptable salt and solvate thereof.

Within the context of the present invention, the terms describing the indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

Within the context of the present invention, the term psychotic disorder includes:—

Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-induced Psychotic Disorder including the subtypes With Delusions (293.81) and With Hallucinations (293.82); and Psychotic Disorder Not Otherwise Specified (298.9).

Compounds of formula (I) and its salts and solvates thereof may also be of use in the treatment of the following disorders:—

Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90):

Anxiety disorders including Social Anxiety Disorder, Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00):

Substance-related disorders including Substance Use Disorders such as Substance Dependence, Substance Craving and Substance Abuse; Substance-induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-induced Mood Disorder, Substance-induced Anxiety Disorder, Substance-induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-induced Mood Disorder, Amphetamine-induced Anxiety Disorder, Amphetamine-induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-induced Psychotic Disorder, Cannabis-induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide:

Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-induced Sleep Disorder including the subtypes Insomnia Type, Hypersomnia Type, Parasomnia Type and Mixed Type:

Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50):

Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23):

Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9):

Enhancement of cognition including the treatment of cognition impairment in other diseases such as schizophrenia, bipolar disorder, depression, other psychiatric disorders and psychotic conditions associated with cognitive impairment, e.g. Alzheimer's disease: and

Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

All of the various forms of the psychotic disorders mentioned herein are contemplated as part of the present invention.

“Treatment” includes prophylaxis, where this is appropriate for the relevant condition(s).

It will be appreciated by those skilled in the art that the compounds of formula (I) or pharmaceutically acceptable salts or solvates thereof according to the invention may advantageously be used in conjunction with one or more other therapeutic agents, for instance, 5HT₃ antagonists, serotonin agonists, NK-1 antagonists, selective serotonin reuptake inhibitors (SSRI), noradrenaline re-uptake inhibitors (SNRI), non-selective reuptake inhibitors of one or more of serotonin, noradrenaline and norepinephrine, CRF-1 antagonists, tricyclic antidepressants, dopaminergic antidepressants, H₃ antagonists, 5HT_{1A} antagonists, 5HT_{1B} antagonists, 5HT_{1D} antagonists, 5HT₄ partial agonists, D1 agonists, M1 agonists, anticonvulsant agents, non-steroidal anti-inflammatory drugs (NSAIDs) and/or cyclooxygenase-2 (COX-2) inhibitors.

Suitable 5HT₃ antagonists which may be used in combination of the compounds of the inventions include for example ondansetron, granisetron and metoclopramide.

Suitable serotonin agonists which may be used in combination with the compounds of the invention include sumatriptan, rauwolscine, yohimbine and metoclopramide.

Suitable SSRIs which may be used in combination with the compounds of the invention include fluoxetine, citalopram, femoxetine, fluvoxamine, paroxetine, indalpine, sertraline and zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the inventions include for example divalproex, carbamazepine and diazepam.

Suitable NSAIDs agents which may be used in combination with the compound of the invention include for example one or more chemical entities selected from ibuprofen, aspirin and its active metabolite salicylate.

Suitable COX-2 inhibitors which may be used in combination with a compound of formula (I) or pharmaceutically acceptable salts or solvates thereof include for example rofecoxib (available under the tradename VIOXX®, from Merck, U.S. Pat. No. 5,474,995); celecoxib (available under the tradename CELEBREX®, from Pfizer, U.S. Pat. No. 5,466,823); valdecoxib (available under the tradename BEXTRA®, from Pfizer, U.S. Pat. No. 6,633,272); etoricoxib (available under the tradename ARCOXIA®, from Merck, U.S. Pat. No. 5,861,419); lumiracoxib (available under the tradename PREXIGE®, from Novartis); paracoxib (U.S. Pat. No. 5,932,598); COX-189 from Novartis; BMS347070 from Bristol Myers Squibb; tiracoxib (JTE522) from Japan Tobacco; ABT963 from Abbott; CS502 from Sankyo; 2-(4-ethoxyphenyl)-3-(3-methanesulfonylphenyl)-pyrazolo[1,5-b]pyridazine (GlaxoSmithKline) and 2-butoxy-4-[4-(methylsulfonyl)phenyl]-6-(trifluoromethyl)pyrimidine (GlaxoSmithKline).

It will be appreciated that the compounds of the combination or composition may be administered simultaneously (either in the same or different pharmaceutical formulations), separately or sequentially.

The compounds of formula (I) and pharmaceutically acceptable salts and solvates thereof are also suitable for combination with other typical and atypical antipsychotics to provide improved treatment of psychotic disorders. Particular advantages associated with the combinations, uses and methods of treatment of compounds of formula (I) and their pharmaceutically acceptable salts and solvates thereof include equivalent or improved efficacy at doses of administration which are lower than those commonly used for the individual components. Improved treatments of positive symptoms and/or negative symptoms and/or cognitive symptoms of the psychotic disorder may also be observed. The combinations, uses and methods of treatment of the invention may also provide advantages in treatment of patients who fail to respond adequately or who are resistant to treatment with certain antipsychotic agents (also known as neuroleptic agents).

The combination therapies of the invention are preferably administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a pharmaceutically acceptable salt or solvate thereof and at least one antipsychotic agent are within the scope of the current invention. In one embodiment of adjunctive therapeutic administration as described herein, a patient is typically stabilised on a therapeutic administration of one or more of the components for a period of time and then receives administration of another component. The compounds of formula (I) or a pharmaceutically acceptable salt or solvate thereof may be administered as adjunctive therapeutic treatment to patients who are receiving administration of at least one antipsychotic agent, but the scope of the invention also includes the adjunctive therapeutic administration of at least one antipsychotic agent to patients who are receiving administration of compounds of formula (I) or a pharmaceutically acceptable salt or solvate thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a pharmaceutically acceptable salt or solvate thereof to a patient receiving therapeutic administration of at least one antipsychotic agent. In a further aspect, the invention provides the use of compounds of formula (I) or a pharmaceutically acceptable salt or solvate thereof in the manufacture of a medicament for adjunctive therapeutic administration for the treatment of a psychotic disorder in a patient receiving therapeutic administration of at least one antipsychotic agent. The invention further provides compounds of formula (I) or a pharmaceutically acceptable salt or solvate thereof for use for adjunctive therapeutic administration for the treatment of a psychotic disorder in a patient receiving therapeutic administration of at least one antipsychotic agent.

In a further aspect, the invention provides a method of treatment of a psychotic disorder by adjunctive therapeutic administration of at least one antipsychotic agent to a patient receiving therapeutic administration of compounds of formula (I) or a pharmaceutically acceptable salt or solvate thereof. In a further aspect, the invention provides the use of at least one antipsychotic agent in the manufacture of a medicament for adjunctive therapeutic administration for the treatment of a psychotic disorder in a patient receiving therapeutic administration of compounds of formula (I) or a pharmaceutically acceptable salt or solvate thereof. The invention further provides at least one antipsychotic agent for adjunctive therapeutic administration for the treatment of a psychotic disorder in a patient receiving therapeutic administration of compounds of formula (I) or a pharmaceutically acceptable salt or solvate thereof.

In a further aspect, the invention provides a method of treatment of a psychotic disorder by simultaneous therapeutic administration of compounds of formula (I) or a pharmaceutically acceptable salt or solvate thereof in combination with at least one antipsychotic agent. The invention further provides the use of a combination of compounds of formula (I) or a pharmaceutically acceptable salt or solvate thereof and at least one antipsychotic agent in the manufacture of a medicament for simultaneous therapeutic administration in the treatment of a psychotic disorder. The invention further provides the use of compounds of formula (I) or a pharmaceutically acceptable salt thereof in the manufacture of a medicament for simultaneous therapeutic administration with at least one antipsychotic agent in the treatment of a psychotic disorder. The invention further provides compounds of formula (I) or a pharmaceutically acceptable salt thereof for use for simultaneous therapeutic administration with at least one antipsychotic agent in the treatment of a psychotic disorder. The invention further provides the use of at least one antipsychotic agent in the manufacture of a medicament for simultaneous therapeutic administration with compounds of formula (I) or a pharmaceutically acceptable salt thereof in the treatment of a psychotic disorder.

In further aspects, the invention provides a method of treatment of a psychotic disorder by simultaneous therapeutic administration of a pharmaceutical composition comprising compounds of formula (I) or a pharmaceutically acceptable salt or solvate thereof and at least one mood stabilising or antimanic agent, a pharmaceutical composition comprising compounds of formula (I) or a pharmaceutically acceptable salt or solvate thereof and at least one mood stabilising or antimanic agent, the use of a pharmaceutical composition comprising compounds of formula (I) or a pharmaceutically acceptable salt or solvate thereof and at least one mood stabilising or antimanic agent in the manufacture of a medicament for the treatment of a psychotic disorder, and a pharmaceutical composition comprising compounds of formula (I) or a pharmaceutically acceptable salt or solvate thereof and at least one mood stabilising or antimanic agent for use in the treatment of a psychotic disorder.

In a further aspect, the invention provides a kit-of-parts for use in the treatment of a psychotic disorder comprising a first dosage form comprising compounds of formula (I) or a pharmaceutically acceptable salt or solvate thereof and one or more further dosage forms each comprising a antipsychotic agent for simultaneous therapeutic administration.

Within the context of the present invention, the term psychotic disorder includes those disorders mentioned above, such as schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, dyskinetic disorders, depression, bipolar disorder, cognitive impairment and obsessive-compulsive disorders and all the various forms of the disorders as mentioned herein, which are contemplated as part of the present invention.

Examples of antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benzisothiazolyl-piperazines; triazine such as lamotrigine; dibenzoxazepines, such as loxapine; dihydroindolones, such as molindone; aripiprazole; and derivatives thereof that have antipsychotic activity.

Examples of tradenames and suppliers of selected antipsychotic drugs that are suitable for use in the present invention are as follows: clozapine (available under the tradename CLOZARIL®, from Mylan, Zenith Goldline, UDL, Novartis); olanzapine (available under the tradename ZYPREXA®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); sertindole (available under the tradename SERLECT®); amisulpride (available under the tradename SOLION®, from Sanofi-Synthelabo); haloperidol (available under the tradename HALDOL®, from Ortho-McNeil); haloperidol decanoate (available under the tradename HALDOL decanoate®); haloperidol lactate (available under the tradenames HALDOL® and INTENSOL®) chlorpromazine (available under the tradename THORAZINE®, from SmithKline Beecham (GSK); fluphenazine (available under the tradename PROLIXIN®, from Apothecon, Copley, Schering, Teva, and American Pharmaceutical Partners, Pasadena); fluphenazine decanoate (available under the tradename PROLIXIN decanoate®); fluphenazine enanthate (available under the tradename PROLIXIN®); fluphenazine hydrochloride (available under the tradename PROLIXIN®); thiothixene (available under the tradename NAVANE®;, from Pfizer); thiothixene hydrochloride (available under the tradename NAVANE®); trifluoperazine (10-[3-(4-methyl-1-piperazinyl)propyl]-2-(trifluoromethyl)phenothiazine dihydrochloride, available under the tradename STELAZINE®, from SmithKlien Beckman; perphenazine (available under the tradename TRILAFON®; from Schering); perphenazine and amitriptyline hydrochloride (available under the tradename ETRAFON TRILAFON®); thioridazine (available under the tradename MELLARIL®; from Novartis, Roxane, HiTech, Teva, and Alpharma); molindone (available under the tradename MOBAN®, from Endo); molindone hydrochloride (available under the tradename MOBAN®); loxapine (available under the tradename LOXITANE®; from Watson); loxapine hydrochloride (available under the tradename LOXITANE®); and loxapine succinate (available under the tradename LOXITANE®). Furthermore, benperidol (Glianimon®), perazine (Taxilan®) or melperone (Eunerpan®)) may be used.

Other suitable antipsychotic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), iloperidone, pimozide and flupenthixol.

In one further aspect of the invention, suitable antipsychotic agents include olanzapine, risperidone, quetiapine, aripiprazole, haloperidol, clozapine, ziprasidone and osanetant.

For use in medicine, the compounds of the present invention are usually administered as a standard pharmaceutical composition. The present invention therefore provides in a further aspect a pharmaceutical composition comprising a compound of formula (I) as hereinbefore described or a pharmaceutically (i.e. physiologically) acceptable salt thereof and a pharmaceutically (i.e. physiologically) acceptable carrier. The pharmaceutical composition can be for use in the treatment of any of the conditions described herein.

The compounds of formula (I) may be administered by any convenient method, for example by oral, parenteral (e.g. intravenous), buccal, sublingual, nasal, rectal or transdermal administration and the pharmaceutical compositions adapted accordingly.

The compounds of formula (I) as hereinbefore described and their pharmaceutically acceptable salts which are active when given orally can be formulated as liquids or solids, for example syrups, suspensions or emulsions, tablets, capsules and lozenges.

A liquid formulation will generally consist of a suspension or solution of the compound or pharmaceutically acceptable salt in a suitable liquid carrier(s) for example an aqueous solvent such as water, ethanol or glycerine, or a non-aqueous solvent, such as polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active substance in a pharmaceutically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas such as compressed air or an organic propellant such as a fluorochlorohydrocarbon. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches. The composition is suitably in unit dose form such as a tablet, capsule or ampoule.

Each dosage unit for oral administration may contain from 1 to 250 mg (and for parenteral administration contains preferably from 0.1 to 25 mg) of a compound of the formula (I) or a pharmaceutically acceptable salt thereof calculated as the free base.

The pharmaceutically acceptable compounds of the invention will normally be administered in a daily dosage regimen (for an adult patient) of, for example, an oral dose of between 1 mg and 250 mg, such as between 1 mg and 250 mg, such as between 2 mg and 100 mg, e.g. between 2 and 50 mg or an intravenous, subcutaneous, or intramuscular dose of between 0.1 mg and 100 mg, preferably between 0.1 mg and 50 mg, e.g. between 1 and 25 mg of the compound of the formula (I) or a pharmaceutically acceptable salt thereof calculated as the free base, the compound being administered 1 to 4 times per day. Suitably the compounds will be administered for a period of continuous therapy, for example for a week or more.

No adverse toxicological effects have been observed for compounds of the invention at doses expected to be approved for therapeutic administration.

7-[4-(4-Chlorobenzyloxy)benzenesulfonyl]-8-methoxy-3-methyl-2,3,4,5-tetrahydro-1H-3-benzazepinium maleate or tosylate will normally be administered in a daily dosage regimen (for an adult patient) of, for example, an oral dose of between 1 mg and 250 mg, such as between 1 mg and 250 mg, such as between 2 mg and 100 mg, e.g. between 2 and 50 mg or an intravenous, subcutaneous, or intramuscular dose of between 0.1 mg and 100 mg, for example between 0.1 mg and 50 mg, e.g. between 1 and 25 mg of 7-[4-(4-chlorobenzyloxy)benzenesulfonyl]-8-methoxy-3-methyl-2,3,4,5-tetrahydro-1H-3-benzazepinium calculated as the free base, the compound being administered 1 to 4 times per day. Suitably the compounds will be administered for a period of continuous therapy, for example for a week or more.

Biological Test Methods

Binding Experiments on Cloned Dopamine (e.g. D2 and D3) Receptors

The ability of the compounds to bind selectively to human D2/D3 dopamine receptors can be demonstrated by measuring their binding to cloned receptors. The inhibition constants (Ki) of test compounds for displacement of [125I]-Iodosulpride binding to human D2/D3 receptors expressed in CHO cells were determined as follows. The cell lines were shown to be free from bacterial, fungal and mycoplasmal contaminants, and stocks of each were stored frozen in liquid nitrogen. Cultures were grown as monolayers or in suspension in standard cell culture media. Cells were recovered by scraping (from monolayers) or by centrifugation (from suspension cultures), and were washed two or three times by suspension in phosphate buffered saline followed by collection by centrifugation. Cell pellets were stored frozen at −80° C. Crude cell membranes were prepared by homogenisation followed by high-speed centrifugation, and characterisation of cloned receptors achieved by radioligand binding.

Preparation of CHO cell membranes: Cell pellets were gently thawed at room temperature, and resuspended in about 20 volumes of ice-cold Extraction buffer; 5 mM EDTA, 50 mM Trizma pre-set crystals (pH7.4@37oC), 1 mM MgCl2, 5 mM KCl and 120 mM NaCl. The suspension was homogenised using an Ultra-Turrax at full speed for 15 seconds. The homogenate was centrifuged at 18,000 r.p.m for 15 min at 4° C. in a Sorvall RC5C centrifuge. Supernatant was discarded, and homogenate re-suspended in extraction buffer then centrifugation was repeated. The final pellet was resuspended in 50 mM Trizma pre-set crystals (pH 7.4 @ 37° C.) and stored in 1 ml aliquot tubes at −80oC (D2=3.0E+08 cells, D3=7.0E+07 cells and D4=1.0E+08 cells). The protein content was determined using a BCA protocol and bovine serum albumin as a standard (Smith, P. K., et al., Measurement of protein using bicinchoninic acid. Anal. Biochem. 150, 76-85 (1985)).

Binding experiments: Crude D2/D3 cell membranes were incubated with 0.03 nM [125I]-Iodosulpride (˜2000 Ci/mmol; Amersham, U. K., and the test compound in a buffer containing 50 mM Trizma pre-set crystals (pH 7.4 @ 37° C.), 120 mM NaCl, 5 mM KCl, 2 mM CaCl2, 1 mM MgCl2, 0.3% (w/v) bovine serum albumin. The total volume is 0.2 ml and incubated in a water bath at 37° C. for 40 minutes. Following incubation, samples were filtered onto GF/B Unifilters using a Canberra Packard Filtermate, and washed four times with ice-cold 50 mM Trizma pre-set crystals (pH 7.4 @ 37oC). The radioactivity on the filters was measured using a Canberra Packard Topcount Scintillation counter. Non-specific binding was defined with 10 μM SKF-102161 (YM-09151). For competition curves, 10 serial log concentrations of competing cold drug were used (Dilution range: 10 μM-10 pM). Competition curves were analysed using Inflexion, an iterative curve fitting programme in Excel. Results were expressed as pKi values where
pKi=−log 10[Ki].

The exemplified compounds have pKᵢ values within the range of 6.8-8.3 at the dopamine D₃ receptor.

The exemplified compounds have pKᵢ values within the range of 6.7-7.9 at the dopamine D₂ receptor.

Binding Experiments on Cloned 5-HT₆ Receptors

Compounds can be tested following the procedures outlined in WO 98/27081.

The exemplified compounds have pKi values within the range of 7.6-8.8 at the serotonin 5-HT₆ receptor.

Binding Experiments on Cloned 5-HT_{2A} and 5-HT_{2C} Receptors

Compounds can be tested following the procedures outlined in WO 94/04533.

The exemplified compounds have pKi values within the range of 7.1-8.4 at the serotonin 5-HT_{2C} receptor and 8.1-9.2 at the serotonin 5-HT_{2A} receptor.

The invention is further illustrated by the following non-limiting examples:

Description 1

### 7-Hydroxy-8-nitro-1,2,4,5-tetrahydro-3-benzazepine-3-carboxylic acid tert-butyl ester (D1)

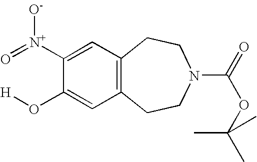

To a solution of 7-hydroxy-1,2,4,5-tetrahydro-3-benzazepine-3-carboxylic acid tert-butyl ester (100 g, 0.38 mol) in dichloromethane (500 ml) was added 69% nitric acid (25 ml) dropwise whilst stirring over an ice-bath to maintain internal reaction temperature of about 25° C. Upon complete addition of acid, cooling was removed and the mixture was stirred at room temperature for 1.5 hours. The reaction mixture was transferred to a separating funnel and washed with water (2×500 ml), then brine/water (250/250 ml). The organic phase was passed through a phase-separation funnel then concentrated in vacuo and the residue was purified by silica chromatography eluting with 0-15% ethyl acetate in hexane as eluants to give the desired product 61.5 g (53%) as a slightly yellow solid. ¹H NMR δ (CDCl₃) 1.48 (s, 9H), 2.86-2.93 (m, 4H), 3.53-3.59 (m, 4H), 6.92 (s, 1H), 7.84 (s, 1H), 10.50 (s, 1H).

Description 2

### 7-Nitro-8-trifluoromethanesulfonyloxy-1,2,4,5-tetrahydro-3-benzazepine-3-carboxylic acid tert-butyl ester (D2)

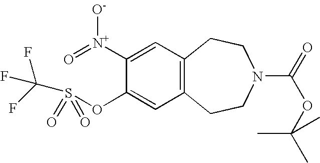

To a solution of D1 (61.5 g, 0.20 mol) and triethylamine (41.2 ml, 0.30 mol) in acetone (1000 ml) at 0° C. was added trifluoromethanesulfonyl chloride (32 ml, 0.30 mol) dropwise, with stirring. Upon complete addition the cooling was removed and the mixture was stirred at room temperature for 1 hour. The reaction mixture was concentrated in vacuo and the residual solid was dissolved in dichloromethane (500 ml) and washed with saturated aqueous sodium bicarbonate solution (500 ml). The organic phase was passed through a phase-separation funnel then concentrated in vacuo to give 88 g (100%) of the title compound. ¹H NMR δ (CDCl₃) 1.48 (s, 9H), 2.93-3.03 (m, 4H), 3.59-3.63 (m, 4H), 7.19 (s, 1H), 7.95 (s, 1H).

Description 3

### 7-(4-Bromo-phenylsulfanyl)-8-nitro-1,2,4,5-tetrahydro-3-benzazepine-3-carboxylic acid tert-butyl ester (D3)

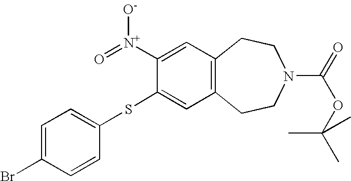

To a solution of D2 (88.0 g, 0.20 mol) and 4-bromobenzenethiol (75.6 g, 0.40 mol) in acetonitrile (900 ml) at 0° C. was added ethyl-diisopropylamine (91 ml, 0.52 mol) dropwise, with stirring. Upon complete addition the cooling was removed and the mixture was stirred at room temperature for 1 hour. The reaction mixture was diluted with toluene (1500 ml) and transferred to a separating funnel and washed with 1M HCl (3000 ml), water (500 ml), then brine (200 ml). The organic phase was concentrated in vacuo and the residual solid was triturated in ethanol (500 ml) then filtered, washing the residue well with ethanol. The resultant yellow solid was dried in vacuo at 50° C. 83.1 g (87%) of the title compound was obtained. ¹H NMR δ (CDCl₃) 1.46 (s, 9H), 2.70-2.75 (m, 2H), 2.89-2.93 (m, 2H), 3.45-3.49 (m, 2H), 3.52-3.56 (m, 2H), 6.57 (s, 1H), 7.41-7.46 (m, 2H), 7.58-7.63 (m, 2H), 8.01 (s, 1H).

Description 4

### 7-Amino-8-(4-bromo-phenylsulfanyl)-1,2,4,5-tetrahydro-3-benzazepine-3-carboxylic acid tert-butyl ester (D4)

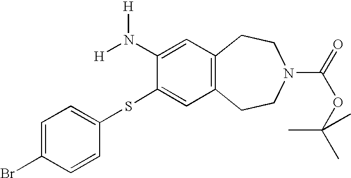

To a solution of D3 (83.1 g, 0.17 mol) and acetic acid (200 ml) in ethanol (1000 ml) was added iron filings (45.4 g). The yellow mixture was heated, with stirring at 110° C. for 5 hours. Upon cooling, the dark resultant mixture was diluted ethyl acetate (500 ml) and water (500 ml), filtered to remove iron residues then transferred to a separating funnel. The phases were separated and the organic phase was further washed with water, saturated aqueous sodium bicarbonate (1000 ml), then brine (500 ml), then passed through a phase separation column and concentrated in vacuo affording 83.0 g (100%) of the title compound as an off-white solid. ¹H NMR δ (CDCl₃) 1.48 (s, 9H), 2.75-2.83 (m, 4H), 3.48-3.57 (m, 4H), 4.13-4.15 (m, 2H), 6.89-6.95 (m, 2H), 7.17 (s, 1H), 7.25-7.34 (m, 2H).

Description 5

### (4-Bromo-phenylsulfanyl)-dimethylamino-1,2,4,5-tetrahydro-3-benzazepine-3-carboxylic acid dimethyl-ethyl ester (D5)

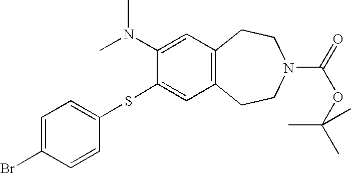

A mixture of D4 (83 g, 0.18 mol), acetic acid (50 ml) and formaldehyde (50 ml, 37% w/w aqueous solution) in dichloromethane (750 ml) was stirred at room temperature for 15 minutes. Sodium triacetoxyborohydride (200 g) was added portionwise over 2 hours, then the mixture stirred for a further 1 hour at room temperature. The resultant mixture was neutralised by careful addition of saturated aqueous sodium bicarbonate and the mixture was transferred to a separating funnel and separated. The organic phase was passed through a phase separation column then concentrated in vacuo to give 88 g (99%) of title compound as an off-white solid. ¹H NMR δ (CDCl₃) 1.47 (s, 9H), 2.68-2.75 (m, 2H), 2.76 (s, 6H), 2.82-2.87 (m, 2H), 3.44-3.50 (m, 2H), 3.51-3.57 (m, 2H), 6.77 (s, 1H), 6.84 (s, 1H), 7.16-7.21 (m, 2H), 7:38-7.44 (m, 2H).

Description 6

### (4-Bromo-benzenesulfonyl)-dimethylamino-1,2,4,5-tetrahydro-3-benzazepine-3-carboxylic acid dimethyl-ethyl ester (D6)

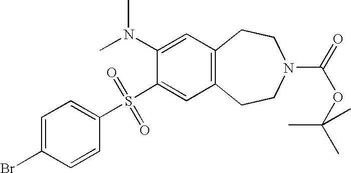

To a suspension of magnesium monoperoxyphthalate hexahydrate (350.0 g 80% pure, 0.55 mol) in dichloromethane (500 ml) and methanol (100 ml) stirring at 0° C. was added D5 (88.0 g, 0.18 mol) in dichloromethane (150 ml) over 15 minutes. Upon complete addition, cooling was removed and the mixture stirred at room temperature for 3 hours. The mixture was carefully quenched at 0° C. with saturated aqueous sodium sulfite solution, until starch-iodide papers indicate negative for oxidant. The mixture was transferred to a separating funnel, diluted with water (1000 ml) and product extracted with dichloromethane (2×500 ml). The combined organic phase washed with brine then passed through a phase separation column and concentrated in vacuo to give 79.4 g (85%) of the title compound as an off-white solid. ¹H NMR δ (CDCl₃) 1.47 (s, 9H), 2.41 (s, 6H), 2.81-2.89 (m, 2H), 2.93-2.98 (m, 2H), 3.54-3.61 (m, 4H), 6.98 (s, 1H), 7.52-7.54 (m, 2H), 7.71-7.78 (m, 2H), 7.94 (s, 1H).

Description 7

### [8-(4-Bromo-benzenesulfonyl)-3-methyl-2,3,4,5-tetrahydro-1-H-3-benzazepin-7-yl]-dimethyl-amine (D7)

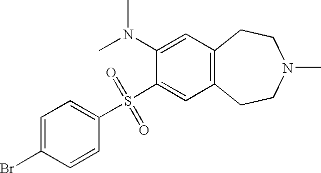

To a stirred solution of D6 (79.4 g, 0.15 mol) in ethanol (200 ml) was added a solution of 4N HCl in dioxan (400 ml) and the mixture stirred at 40° C. for 2 hours. Solvents were removed in vacuo and the resultant sticky gum was triturated with ether and the ether decanted off to remove excess HCl. To the free flowing off-white solid was added 1,2-dichloroethane (700 ml) and formaldehyde (100 ml, 37% w/w aqueous solution), and the mixture stirred for 20 minutes at room temperature before addition of sodium triacetoxyborohydride (75.0 g) portionwise over 45 minutes. Upon complete addition, the mixture was diluted with dichloromethane (500 ml) and washed carefully with saturated aqueous sodium bicarbonate (1000 ml). The organic phase was passed through a phase separation column and concentrated in vacuo and the resultant gum was purified by silica chromatography eluting with 0-3% methanol in dichloromethane to give 53.4 g (81%) of the title compound as an off-white solid. ¹H NMR δ (CDCl₃) 2.38 (s, 3H), 2.40 (s, 6H), 2.54-2.62 (m, 4H), 2.90-2.94 (m, 2H), 2.97-3.01 (m, 2H), 6.96 (s, 1H), 7.54-7.60 (m, 2H), 7.73-7.78 (m, 2H), 7.92 (s, 1H).

### EXAMPLE 1

### {8-[4-(3-Fluoro-5-trifluoromethyl-benzyl)-benzenesulfonyl]-3-methyl-2,3,4,5-tetrahydro-1H-3-benzazepin-7-yl}-dimethyl-amine (E1)

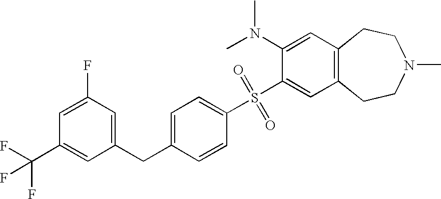

An oven dried 3-neck flask was charged with cut zinc foil (0.30 g, 4.6 mmol) under an argon atmosphere followed by anhydrous tetrahydrofuran (1 ml) and 1,2-dibromobutane (20 μl). The mixture was heated to reflux with a hot air gun for one minute then cooled to room temperature. This action was repeated a further two times, darkening the zinc foil, before the addition of chlorotrimethylsilane (20 μl) at room temperature. After 15 minutes stirring at room temperature the mixture was cooled to 0° C. and a solution of 3-fluoro-5-trifluoromethylbenzyl bromide (1.0 g, 3.9 mmol) in anhydrous tetrahydrofuran (1.0 ml) was added over 40 minutes. The mixture was then allowed to warm to room temperature and stirred for 2 hours at room temperature. The solution was added at room temperature to a mixture of D7 (200 mg, 0.47 mmol) and tetrakis(triphenylphosphine) palladium (27.0 mg, 0.02 mmol) in anhydrous tetrahydrofuran (1.0 ml) leaving zinc foil behind. The resultant mixture was stirred at 70° C. for 2 h, then cooled and applied to an SCX cartridge and eluted with methanol then 5% ammonia in methanol. The 5% ammonia in methanol fractions were combined and concentrated in vacuo and the residue was purified by silica chromatography eluting with 0-5% methanol in dichloromethane to give 170 mg (70%) of the title compound as an off-white solid. MH⁺ 521. ¹H NMR δ (CDCl₃) 2.35 (s, 6H), 2.38 (s, 3H), 2.54-2.62 (m, 4H), 2.89-2.94 (m, 2H), 2.97-3.01 (m, 2H), 4.07 (s, 2H), 6.96-7.01 (m, 2H), 7.14-7.27 (m, 4H), 7.828-7.87 (m, 2H), 7.94 (s, 1H).

Examples 2-55 were prepared using analogous procedures to Example 1 using the appropriate benzyl bromide. Products were isolated as either the free base or monohydrochloride salt. All ¹H NMR are consistent with the structures shown.

All of the compounds listed below in Table 1 relate to compounds of formula (I) as described above).

TABLE 1

| | |
|---|---|
| | (I) |
| 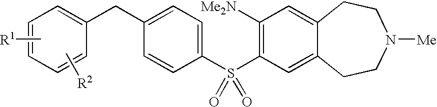 | |

| Example No. | R¹ | R² |
|---|---|---|
| 1 | 5-CF₃ | 3-F |
| 2 | 4-CN | H |
| 3 | 3-CF₃ | H |
| 4 | 2-Cl | 4-Cl |
| 5 | 4-OEt | H |
| 6 | 3-CF₃ | 4-F |
| 7 | 2-OMe | H |
| 8 | 3-F | H |
| 9 | 4-SO₂Me | H |
| 10 | 4-CF₃ | 3-OMe |
| 11 | 3-F | 5-F |
| 12 | 5-CF₃ | 2-F |
| 13 | 2-CF₃ | 4-F |
| 14 | 3-CF₃ | 2-Cl |
| 15 | 2-CF₃ | 5-F |
| 16 | 5-CF₃ | 2-Cl |
| 17 | 2-F | 4-F |
| 18 | 2-Cl | 4-F |
| 19 | 3-Cl | 4-F |
| 20 | 3-OCF₃ | H |
| 21 | 2-F | 5-F |
| 22 | 3-Me | 2-F |
| 23 | 2-Me | 5-F |
| 24 | 3-Me | 4-F |
| 25 | 3-Cl | 5-F |
| 26 | 4-CF₃ | 3-F |
| 27 | 3-F | 4-F |
| 28 | 3-OMe | H |
| 29 | 4-OMe | H |
| 30 | 2-Me | H |
| 31 | 3-Me | H |
| 32 | 4-Me | H |
| 33 | 2-Cl | H |
| 34 | 3-Cl | H |
| 35 | 4-Cl | H |
| 36 | 2-F | H |
| 37 | 3-CN | H |
| 38 | 3-Cl | 4-Cl |
| 39 | 4-Me | 3-F |
| 40 | 2-CN | H |
| 41 | 4-OCF₃ | H |
| 42 | 3-CF₃ | 5-F |
| 43 | 2-Cl | 4-CN |
| 44 | 2-F | 4-CN |
| 45 | 4-CF₃ | 2-F |
| 46 | 3-CF₃ | 4-Cl |
| 47 | 2-CF₃ | 4-CN |
| 48 | 4-(5-Me-1,2,4- | H |
| | oxadiazol-3-yl) | |
| 49 | 5-F | 3-CN |
| 50 | 4-F | 2-CN |
| 51 | 4-F | 3-CN |
| 52 | 3-F | 4-CN |
| 53 | 4-SO₂NMe2 | H |
| 54 | 3-CN | 4-OMe |
| 55 | 4- | H |
| | SO₂morpholinyl | |

All publications, including but not limited to patents and patent applications, cited in this specification are herein incorporated by reference as if each individual publication were specifically and individually indicated to be incorporated by reference herein as though fully set forth.

1. One or more chemical entities selected from a compound of formula (I): 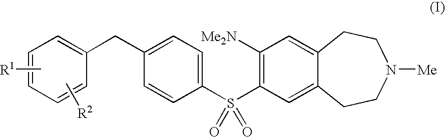
wherein
R¹ represents C₁₋₆alkyl, C₁₋₆alkoxy, trifluoromethyl, trifluoromethoxy, halo, cyano, 5-methyl-1,2,4-oxadiazol-3-yl or a group —SO₂X;
R² represents hydrogen, C₁₋₆alkyl, C₁₋₆alkoxy, halo or cyano;
X is C₁₋₆alkyl, —NR³R⁴ or morpholino;
R³ and R⁴ independently represent hydrogen or C₁₋₆alkyl;
and a pharmaceutically acceptable salt and solvate thereof;
with the proviso that the compound {8-[4-(4-fluoro-benzyl)-benzenesulfonyl]-3-methyl-2,3,4,5-tetrahydro-1H-3-benzazepin-7-yl}-dimethyl-amine is excluded.

2. One or more chemical entities as claimed in claim 1 selected from
{8-[4-(3-Fluoro-5-trifluoromethyl-benzyl)-benzenesulfonyl]-3-methyl-2,3,4,5-tetrahydro-1H-3-benzazepin-7-yl}-dimethyl-amine;
{8-[4-(4-Cyanobenzyl)benzenesulfonyl]-3-methyl-2,3,4,5-tetrahydro-1H-3-benzazepin-7-yl}-dimethylamine;
{8-[4-(2,4-Dichlorobenzyl)benzenesulfonyl]-3-methyl-2,3,4,5-tetrahydro-1H-3-benzazepin-7-yl}-dimethylamine;
{8-[4-(3-Trifluoromethylbenzyl)benzenesulfonyl]-3-methyl-2,3,4,5-tetrahydro-1H-3-benzazepin-7-yl}-dimethylamine;
{8-[4-(4-Ethoxybenzyl)benzenesulfonyl]-3-methyl-2,3,4,5-tetrahydro-1H-3-benzazepin-7-yl}-dimethylamine;
{8-[4-(4-Fluoro-3-trifluoromethylbenzyl)benzenesulfonyl]-3-methyl-2,3,4,5-tetrahydro-1H-3-benzazepin-7-yl}-dimethylamine;
{8-[4-(2-Methoxybenzyl)benzenesulfonyl]-3-methyl-2,3,4,5-tetrahydro-1H-3-benzazepin-7-yl}-dimethylamine;
{8-[4-(3-Fluorobenzyl)benzenesulfonyl]-3-methyl-2,3,4,5-tetrahydro-1H-3-benzazepin-7-yl}-dimethylamine;
{8-[4-(4-Methanesulfonylbenzyl)benzenesulfonyl]-3-methyl-2,3,4,5-tetrahydro-1H-3-benzazepin-7-yl}-dimethylamine;
{8-[4-(3-Methoxy-4-trifluoromethylbenzyl)benzenesulfonyl]-3-methyl-2,3,4,5-tetrahydro-1H-3-benzazepin-7-yl}-dimethylamine;
{8-[4-(3,5-Difluorobenzyl)benzenesulfonyl]-3-methyl-2,3,4,5-tetrahydro-1H-3-benzazepin-7-yl}-dimethylamine;
{8-[4-(2-Fluoro-5-trifluoromethylbenzyl)benzenesulfonyl]-3-methyl-2,3,4,5-tetrahydro-1H-3-benzazepin-7-yl}-dimethylamine;
{8-[4-(2-Trifluoromethyl-4-fluorobenzyl)benzenesulfonyl]-3-methyl-2,3,4,5-tetrahydro-1H-3-benzazepin-7-yl}-dimethylamine;
{8-[4-(2-Chloro-3-trifluoromethylbenzyl)benzenesulfonyl]-3-methyl-2,3,4,5-tetrahydro-1H-3-benzazepin-7-yl}-dimethylamine;
{8-[4-(2-Trifluoromethyl-5-fluorobenzyl)benzenesulfonyl]-3-methyl-2,3,4,5-tetrahydro-1H-3-benzazepin-7-yl}-dimethylamine;
{8-[4-(2-Chloro-5-trifluoromethylbenzyl)benzenesulfonyl]-3-methyl-2,3,4,5-tetrahydro-1H-3-benzazepin-7-yl}-dimethylamine;
{8-[4-(2,4-Difluorobenzyl)benzenesulfonyl]-3-methyl-2,3,4,5-tetrahydro-1H-3-benzazepin-7-yl}-dimethylamine;
{8-[4-(2-Chloro-4-fluorobenzyl)benzenesulfonyl]-3-methyl-2,3,4,5-tetrahydro-1H-3-benzazepin-7-yl}-dimethylamine;
{8-[4-(3-Chloro-4-fluorobenzyl)benzenesulfonyl]-3-methyl-2,3,4,5-tetrahydro-1H-3-benzazepin-7-yl}-dimethylamine;
{8-[4-(3-Trifluoromethoxybenzyl)benzenesulfonyl]-3-methyl-2,3,4,5-tetrahydro-1H-3-benzazepin-7-yl}-dimethylamine;
{8-[4-(2,5-Difluorobenzyl)benzenesulfonyl]-3-methyl-2,3,4,5-tetrahydro-1H-3-benzazepin-7-yl}-dimethylamine;
{8-[4-(2-Fluoro-3-methylbenzyl)benzenesulfonyl]-3-methyl-2,3,4,5-tetrahydro-1H-3-benzazepin-7-yl}-dimethylamine;
{8-[4-(2-Methyl-5-fluorobenzyl)benzenesulfonyl]-3-methyl-2,3,4,5-tetrahydro-1H-3-benzazepin-7-yl}-dimethylamine;
{8-[4-(3-Methyl-4-fluorobenzyl)benzenesulfonyl]-3-methyl-2,3,4,5-tetrahydro-1H-3-benzazepin-7-yl}-dimethylamine;
{8-[4-(3-Chloro-5-fluorobenzyl)benzenesulfonyl]-3-methyl-2,3,4,5-tetrahydro-1H-3-benzazepin-7-yl}-dimethylamine;
{8-[4-(3-Fluoro-4-trifluoromethylbenzyl)benzenesulfonyl]-3-methyl-2,3,4,5-tetrahydro-1H-3-benzazepin-7-yl}-dimethylamine;
{8-[4-(3,4-Difluorobenzyl)benzenesulfonyl]-3-methyl-2,3,4,5-tetrahydro-1H-3-benzazepin-7-yl}-dimethylamine;
{8-[4-(3-Methoxybenzyl)benzenesulfonyl]-3-methyl-2,3,4,5-tetrahydro-1H-3-benzazepin-7-yl}-dimethylamine;
{8-[4-(4-Methoxybenzyl)benzenesulfonyl]-3-methyl-2,3,4,5-tetrahydro-1H-3-benzazepin-7-yl}-dimethylamine;
{8-[4-(2-Methylbenzyl)benzenesulfonyl]-3-methyl-2,3,4,5-tetrahydro-1H-3-benzazepin-7-yl}-dimethylamine;
{8-[4-(3-Methylbenzyl)benzenesulfonyl]-3-methyl-2,3,4,5-tetrahydro-1H-3-benzazepin-7-yl}-dimethylamine;
{8-[4-(4-Methylbenzyl)benzenesulfonyl]-3-methyl-2,3,4,5-tetrahydro-1H-3-benzazepin-7-yl}-dimethylamine;
{8-[4-(2-Chlorobenzyl)benzenesulfonyl]-3-methyl-2,3,4,5-tetrahydro-1H-3-benzazepin-7-yl}-dimethylamine;
{8-[4-(3-Chlorobenzyl)benzenesulfonyl]-3-methyl-2,3,4,5-tetrahydro-1H-3-benzazepin-7-yl}-dimethylamine;
{8-[4-(4-Chlorobenzyl)benzenesulfonyl]-3-methyl-2,3,4,5-tetrahydro-1H-3-benzazepin-7-yl}-dimethylamine;
{8-[4-(2-Fluorobenzyl)benzenesulfonyl]-3-methyl-2,3,4,5-tetrahydro-1H-3-benzazepin-7-yl}-dimethylamine;
{8-[4-(3-Cyanobenzyl)benzenesulfonyl]-3-methyl-2,3,4,5-tetrahydro-1H-3-benzazepin-7-yl}-dimethylamine;
{8-[4-(3,4-Dichlorobenzyl)benzenesulfonyl]-3-methyl-2,3,4,5-tetrahydro-1H-3-benzazepin-7-yl}-dimethylamine;
{8-[4-(3-Fluoro-4-methylbenzyl)benzenesulfonyl]-3-methyl-2,3,4,5-tetrahydro-1H-3-benzazepin-7-yl}-dimethylamine;
{8-[4-(2-Cyanobenzyl)benzenesulfonyl]-3-methyl-2,3,4,5-tetrahydro-1H-3-benzazepin-7-yl}-dimethylamine;
{8-[4-(4-Trifluoromethoxybenzyl)benzenesulfonyl]-3-methyl-2,3,4,5-tetrahydro-1H-3-benzazepin-7-yl}-dimethylamine;
{8-[4-(3-Trifluoromethyl-5-fluorobenzyl)benzenesulfonyl]-3-methyl-2,3,4,5-tetrahydro-1H-3-benzazepin-7-yl}-dimethylamine;
{8-[4-(2-Chloro-4-cyanobenzyl)benzenesulfonyl]-3-methyl-2,3,4,5-tetrahydro-1H-3-benzazepin-7-yl}-dimethylamine;
{8-[(4-(2-Fluoro-4-cyanobenzyl)benzenesulfonyl]-3-methyl-2,3,4,5-tetrahydro-1H-3-benzazepin-7-yl}-dimethylamine;
{8-[4-(4-Trifluoromethyl-2-fluorobenzyl)benzenesulfonyl]-3-methyl-2,3,4,5-tetrahydro-1H-3-benzazepin-7-yl}-dimethylamine;
{8-[4-(3-Trifluoromethyl-4-chlorobenzyl)benzenesulfonyl]-3-methyl-2,3,4,5-tetrahydro-1H-3-benzazepin-7-yl}-dimethylamine;
{8-[4-(2-Trifluoromethyl-4-cyanobenzyl)benzenesulfonyl]-3-methyl-2,3,4,5-tetrahydro-1H-3-benzazepin-7-yl}-dimethylamine;
{8-[4-(5-Methyl-1,2,4-oxadiazol-3-ylbenzyl)benzenesulfonyl]-3-methyl-2,3,4,5-tetrahydro-1H-3-benzazepin-7-yl}-dimethylamine;
3-[(4-{[8-(Dimethylamino)-3-methyl-2,3,4,5-tetrahydro-1H-3-benzazepin-7-yl]sulfonyl}phenyl)methyl]-5-fluorobenzonitrile;
2-[(4-{[8-(Dimethylamino)-3-methyl-2,3,4,5-tetrahydro-1H-3-benzazepin-7-yl]sulfonyl}phenyl)methyl]-5-fluorobenzonitrile;
5-[(4-{[8-(Dimethylamino)-3-methyl-2,3,4,5-tetrahydro-1H-3-benzazepin-7-yl]sulfonyl}phenyl)methyl]-2-fluorobenzonitrile;
4-[(4-([8-(Dimethylamino)-3-methyl-2,3,4,5-tetrahydro-1H-3-benzazepin-7-yl]sulfonyl)phenyl)methyl]-2-fluorobenzonitrile;
4-[(4-{[8-(Dimethylamino)-3-methyl-2,3,4,5-tetrahydro-1H-3-benzazepin-7-yl]sulfonyl}phenyl)methyl]-N,N-dimethylbenzenesulfonamide;
5-[(4-{[8-(Dimethylamino)-3-methyl-2,3,4,5-tetrahydro-1H-3-benzazepin-7-yl]sulfonyl}phenyl)methyl]-2-(methyloxy)benzonitrile and
N,N,3-Trimethyl-8-[(4-{[4-(4-morpholinylsulfonyl)phenyl]methyl}phenyl)sulfonyl]-2,3,4,5-tetrahydro-1H-3-benzazepin-7-amine,
and pharmaceutically acceptable salts and solvates thereof.

3. A pharmaceutical composition comprising one or more chemical entities as claimed in claim 1 and a pharmaceutically acceptable carrier.

4-6. (canceled)

7. A method of treatment of a psychotic disorder in mammals including humans, which comprises administering to a mammal in need thereof an effective amount of one or more chemical entities as claimed in claim 1.

8. A pharmaceutical composition comprising one or more chemical entities as claimed in claim 2 and a pharmaceutically acceptable carrier.

9. A method of treatment of a psychotic disorder in mammals including humans, which comprises administering to a mammal in need thereof an effective amount of one or more chemical entities as claimed in claim 2.
